(19) United States
(12) Patent Application Publication
(10) Pub. No.: US 2025/0311939 A1
(43) Pub. Date: Oct. 9, 2025

(54) SENSOR ARRANGEMENT FOR A MAGNETIC MARKER LOCALIZATION SYSTEM AND METHOD OF DETERMINING A DISPOSITION OF A MAGNETIC MARKER

(71) Applicant: Stryker Corporation, Portage, MI (US)

(72) Inventors: Ananth RAVI, Toronto (CA); John DILLON, Mississauga (CA); Mark SEMPLE, Toronto (CA); Prashant PANDEY, Toronto (CA); Michael GIANNINI, Toronto (CA)

(73) Assignee: Stryker Corporation, Portage, MI (US)

(21) Appl. No.: 19/097,687

(22) Filed: Apr. 1, 2025

Related U.S. Application Data
(60) Provisional application No. 63/574,902, filed on Apr. 4, 2024.
(60) Provisional application No. 63/574,869, filed on Apr. 4, 2024.

(51) Int. Cl. A61B 5/06

(52) U.S. Cl. CPC A61B 5/062

(57) ABSTRACT

The present disclosure may be embodied as a probe for determining a position and pose of an anisotropic magnetic marker having a known size, shape, and magnetization. The probe has a substrate having a first side, a second side, a longitudinal axis, and a transverse axis. A first magnetic sensor is on the first side of the substrate. A second magnetic sensor is on the first side of the substrate and spaced apart from the first magnetic sensor along the longitudinal axis of the substrate and spaced apart from the first magnetic sensor along the transverse axis of the substrate. A third magnetic sensor is on the second side of the substrate. Each of the first magnetic sensor, the second magnetic sensor, and the third magnetic sensor is a multidimensional magnetic sensor. A processor is configured to determine a disposition of the magnetic marker in five degrees of freedom.

### CROSS-REFERENCE TO RELATED APPLICATIONS

This application claims the benefit of U.S. Provisional Application 63/574,869 filed on Apr. 4, 2024, and U.S. Provisional Application 63/574,902 filed on Apr. 4, 2024, the entire contents of which are incorporated herein by reference for all purposes.

### FIELD OF THE DISCLOSURE

The present disclosure relates to localization of markers, and in particular, determining a location and pose of a magnetic marker.

### BACKGROUND OF THE DISCLOSURE

Surgery and other medical procedures/therapies often require accurate localization of an area of interest. Despite advances in modalities and sensors, typical localization techniques involve the use of large sensor probes to accurately localize a wire, seed, or marker. Thus, there is a need for probes which use optimized sensor position so as to decrease a size of the probe, while at the same time allowing for localization of magnetic marker in five degrees of freedom.

### BRIEF SUMMARY OF THE DISCLOSURE

The present disclosure may be embodied as a probe for determining a position and pose of an anisotropic magnetic marker having a known size, shape, and magnetization. The probe has a substrate having a first side, a second side, a longitudinal axis, and a transverse axis. In some embodiments, the substrate has a thickness of between 0.5 mm and 10 mm. A first magnetic sensor is disposed on the first side of the substrate. A second magnetic sensor is disposed on the first side of the substrate and spaced apart from the first magnetic sensor along the longitudinal axis of the substrate and spaced apart from the first magnetic sensor along the transverse axis of the substrate.

A third magnetic sensor is disposed on the second side of the substrate. Each of the first magnetic sensor, the second magnetic sensor, and the third magnetic sensor is a multidimensional magnetic sensor. The third magnetic sensor may be spaced apart from the first magnetic sensor along the longitudinal axis. The third magnetic sensor may be spaced apart from the first magnetic sensor along the transverse axis. The third magnetic sensor may be spaced apart from the first magnetic sensor along the longitudinal axis and the transverse axis. The third magnetic sensor may be spaced apart from the second magnetic sensor along the longitudinal axis. The third magnetic sensor may be spaced apart from the second magnetic sensor along the transverse axis. The third magnetic sensor may be spaced apart from the second magnetic sensor along the longitudinal axis and the transverse axis.

A processor is in electronic communication with the first magnetic sensor, the second magnetic sensor, and the third magnetic sensor. The processor is configured to (e.g., programmed to) determine a disposition of the magnetic marker in five degrees of freedom based on the known size, shape, and magnetization (of the magnetic marker) and signals received from each of the first, second, and third magnetic sensors.

In some embodiments, the spacing between the first and second magnetic sensors is greater along the longitudinal axis than the spacing between the first and second magnetic sensors along the transverse axis.

In some embodiments, at least one of the first magnetic sensor, the second magnetic sensor, and the third magnetic sensor is oriented such that no measurement axis (of such magnetic sensor(s)) is parallel with the longitudinal axis of the substrate. In some embodiments, at least one of the first magnetic sensor, the second magnetic sensor, and the third magnetic sensor has a different orientation from the other magnetic sensors.

In some embodiments, the probe may have a fourth magnetic sensor. The fourth magnetic sensor may be disposed on the first side of the substrate. The fourth magnetic sensor may be disposed on the second side of the substrate. The fourth magnetic sensor may be spaced apart from the third magnetic sensor along the longitudinal axis. The fourth magnetic sensor may be spaced apart from the third magnetic sensor along the transverse axis. The fourth magnetic sensor may be spaced apart from the third magnetic sensor along the longitudinal axis and the transverse axis.

In some embodiments, a maximum total spacing between the first magnetic sensor, the second magnetic sensor, and the third magnetic sensor along the transverse axis is less than or equal to 12 mm. In some embodiments, a maximum total spacing between the first magnetic sensor, the second magnetic sensor, and the third magnetic sensor along the transverse axis is 12 mm, and along the longitudinal axis is between 1.25 and 10 times the maximum total spacing along the longitudinal axis.

The probe may further include a user interface in electronic communication with the processor. The processor may be further configured to provide a signal of the determined disposition of the magnetic marker to the user interface. The user interface may be a monitor configured to display the determined disposition of the magnetic marker according to the signal provided from the processor. The user interface may be an audio source configured to audibly represent the determined disposition of the magnetic marker according to the signal provided from the processor.

The processor may have a first mode in which the disposition of the magnetic marker is determined in five degrees of freedom and a second mode wherein the disposition of the magnetic marker is determined using one of the first magnetic sensor, the second magnetic sensor, or the third magnetic sensor.

The processor may be configured to determine more than one disposition of the magnetic marker over time. The processor may be configured to periodically determine the disposition of the magnetic marker at a sampling frequency.

In some embodiments, the substrate is contained within a probe housing. The magnetic sensors (i.e., first magnetic sensor, second magnetic sensor, etc.) may be contained within the probe housing. The processor may be located outside the probe housing.

The processor may be further configured to provide an indicator signal when magnetic field gradients which are not consistent with the magnetic marker are detected. The processor may be further configured to disregard magnetic field gradients which are not consistent with the magnetic marker.

In some embodiments, one of the first magnetic sensor, the second magnetic sensor, or the third magnetic sensor is spaced apart from the other magnetic sensors along the longitudinal axis and configured to measure a background magnetic field.

In some embodiments, the probe has a background magnetic sensor spaced apart from the first magnetic sensor, the second magnetic sensor, and the third magnetic sensor, along the longitudinal axis, and configured to measure a background magnetic field.

An exemplary probe for determining a disposition of a magnetic marker comprises: a substrate having a first side, a second side, a longitudinal axis, and a transverse axis; a first magnetic sensor disposed on the substrate; a second magnetic sensor neighboring to the first magnetic sensor, the second magnetic sensor disposed on the substrate and spaced further away from a distal end of the substrate than the first magnetic sensor; a third magnetic sensor neighboring to the second magnetic sensor, the third magnetic sensor disposed on the substrate and spaced further away from the distal end of the substrate than the second magnetic sensor, wherein one of the first magnetic sensor, the second magnetic sensor, and the third magnetic sensor is on the first side of the substrate, wherein the other two of the first magnetic sensor, the second magnetic sensor, and the third magnetic sensor are on the second side of the substrate, and wherein measurement axes of each of the first magnetic sensor, the second magnetic sensor, and the third magnetic sensor are not co-linear with corresponding measurement axes of the other magnetic sensors; and a processor in electronic communication with the first magnetic sensor, the second magnetic sensor, and the third magnetic sensor, wherein the processor is configured to determine the disposition of the magnetic marker based on signals received from each of the first, second, and third magnetic sensors.

In some examples, the distance between the first magnetic sensor and the second magnetic sensor along the longitudinal axis of the probe is smaller than the distance between the second magnetic sensor and the third magnetic sensor along the longitudinal axis of the probe. In some examples, the distance between neighboring magnetic sensors along the longitudinal axis of the probe decreases toward the distal end of the probe.

In some examples, the probe further comprises one or more additional magnetic sensors, wherein every two neighboring magnetic sensors on the probe are located on alternating sides of the substrate. In some examples, one of the first magnetic sensor, the second magnetic sensor, and the third magnetic sensor is disposed on a first side of the longitudinal axis of the substrate, and wherein the other two of the first magnetic sensor, the second magnetic sensor, and the third magnetic sensor are disposed on a second side of the longitudinal axis of the substrate.

In some examples, the probe further comprises one or more additional magnetic sensors, wherein every two neighboring magnetic sensors on the probe are located on different sides of the longitudinal axis of the substrate. In some examples, the substrate comprises a straight section and an angled section, and wherein at least one of the magnetic sensors on the probe is located on the angled section. In some examples, the spacing between the first and second magnetic sensors is smaller along the longitudinal axis than the spacing between the first and second magnetic sensors along the transverse axis.

In some examples, a maximum total spacing between the first magnetic sensor, the second magnetic sensor, and the third magnetic sensor along the transverse axis is less than or equal to 12 mm. In some examples, a maximum total spacing between the first magnetic sensor, the second magnetic sensor, and the third magnetic sensor along the longitudinal axis is between 1.25 and 10 times the maximum total spacing along the longitudinal axis.

In some examples, the probe further comprises a user interface in electronic communication with the processor, and wherein the processor is further configured to provide a signal of the determined disposition of the magnetic marker to the user interface. In some examples, the processor has a first mode in which the disposition of the magnetic marker is determined in five degrees of freedom and a second mode wherein the disposition of the magnetic marker is determined using one of the first magnetic sensor, the second magnetic sensor, or the third magnetic sensor.

In some examples, the processor is configured to determine more than one disposition of the magnetic marker over time. In some examples, the processor is configured to periodically determine the disposition of the magnetic marker at a sampling frequency.

In some examples, the substrate is contained within a probe housing. In some examples, the processor is located outside the probe housing.

In some examples, the processor is further configured to provide an indicator signal when magnetic field gradients which are not consistent with the magnetic marker are detected. In some examples, the processor is further configured to disregard magnetic field gradients which are not consistent with the magnetic marker.

In some examples, at least one of the first magnetic sensor, the second magnetic sensor, and the third magnetic sensor is configured to measure a background magnetic field. In some examples, the probe further comprises a background magnetic sensor spaced apart from the first magnetic sensor, the second magnetic sensor, and the third magnetic sensor, along the longitudinal axis, and configured to measure a background magnetic field.

An exemplary method for determining a disposition of a magnetic marker comprises receiving a plurality of magnetic field strength values of the magnetic marker from a plurality of magnetic sensors of a probe, wherein the plurality of magnetic sensors have a pre-determined configuration relative to one another on the probe; generating a plurality of input values based on the plurality of magnetic field strength values; and providing the plurality of input values to a machine-learning model to obtain the disposition of the marker, wherein the machine-learning model is trained using training data generated by one or more training probes having magnetic sensors in the same pre-determined configuration.

In some examples, the machine-learning classifier is neural network. In some examples, the machine-learning model is configured to determine the disposition of the magnetic marker in more than three degrees of freedom. In some examples, the machine-learning model is configured to determine the disposition of the magnetic marker in five degrees of freedom. In some examples, one or more of the plurality of magnetic sensors is a multidimensional magnetic sensor.

In some examples, the magnetic field strength values obtained from the one or more multidimensional magnetic sensors are arranged in at least one vector. In some examples, generating the plurality of input values comprises subtracting a hard iron offset from at least one magnetic field strength value of the plurality of magnetic field strength values.

In some examples, generating the plurality of input values comprises calculating a differential field by subtracting one or more magnetic field strength values collected by a first magnetic sensor of the plurality of magnetic sensors from one or more magnetic field strength values collected by a second magnetic sensor of the plurality of magnetic sensors. In some examples, the first magnetic sensor and the second magnetic sensor are neighboring sensors on the probe.

In some examples, the machine-learning model is configured to be retrained iteratively. In some examples, the machine-learning model is trained using training data collected for one or more training magnetic markers producing magnetic fields having the same anisotropic geometry as the magnetic marker.

In some examples, the probe comprises a substrate having a first side, a second side, a longitudinal axis, and a transverse axis, and wherein the plurality of magnetic sensors comprises a first magnetic sensor disposed on the substrate; a second magnetic sensor neighboring to the first magnetic sensor, the second magnetic sensor disposed on the substrate and spaced further away from a distal end of the substrate than the first magnetic sensor; and a third magnetic sensor neighboring to the second magnetic sensor, the third magnetic sensor disposed on the substrate and spaced further away from the distal end of the substrate than the second magnetic sensor.

In some examples, the measurement axes of each of the first magnetic sensor, the second magnetic sensor, and the third magnetic sensor are not co-linear with corresponding measurement axes of the other magnetic sensors. In some examples, the spacing between the first and second magnetic sensors is smaller along the longitudinal axis than the spacing between the first and second magnetic sensors along the transverse axis. In some examples, the method further comprises displaying the determined disposition of the magnetic marker.

An exemplary non-transitory computer-readable storage medium stores one or more programs, the one or more programs comprising instructions, which when executed by one or more processors of an electronic device having a display, cause the electronic device to: receive a plurality of magnetic field strength values of the magnetic marker from a plurality of magnetic sensors of a probe, wherein the plurality of magnetic sensors have a pre-determined configuration relative to one another on the probe; generate a plurality of input values based on the plurality of magnetic field strength values; and provide the plurality of input values to a machine-learning model to obtain the disposition of the marker, wherein the machine-learning model is trained using training data generated by one or more training probes having magnetic sensors in the same pre-determined configuration.

An exemplary electronic device comprises: one or more processors, a memory, and one or more programs, wherein the one or more programs are stored in the memory and configured to be executed by the one or more processors, the one or more programs including instructions for: receiving a plurality of magnetic field strength values of the magnetic marker from a plurality of magnetic sensors of a probe, wherein the plurality of magnetic sensors have a pre-determined configuration relative to one another on the probe; generating a plurality of input values based on the plurality of magnetic field strength values; and providing the plurality of input values to a machine-learning model to obtain the disposition of the marker, wherein the machine-learning model is trained using training data generated by one or more training probes having magnetic sensors in the same pre-determined configuration.

An exemplary computer program product comprises instructions, which when executed by one or more processors of an electronic device having a display, cause the electronic device to: receive a plurality of magnetic field strength values of the magnetic marker from a plurality of magnetic sensors of a probe, wherein the plurality of magnetic sensors have a pre-determined configuration relative to one another on the probe; generate a plurality of input values based on the plurality of magnetic field strength values; and provide the plurality of input values to a machine-learning model to obtain the disposition of the marker, wherein the machine-learning model is trained using training data generated by one or more training probes having magnetic sensors in the same pre-determined configuration.

It will be appreciated that any of the aspects, features and options described herein can be combined. Any of the aspects, features and options described in view of the probe apply equally to the method, non-transitory computer-readable storage medium, electronic device, and computer program product, and vice versa.

### DESCRIPTION OF THE DRAWINGS

For a fuller understanding of the nature and objects of the disclosure, reference should be made to the following detailed description taken in conjunction with the accompanying drawings.

FIG. 1 is a diagram showing an embodiment of a probe according to an embodiment of the present disclosure.

FIG. 2 is a diagram showing another embodiment of a probe according to an embodiment of the present disclosure.

FIG. 3 is a diagram showing another embodiment of a probe according to an embodiment of the present disclosure.

FIG. 4 is a diagram showing another embodiment of a probe according to an embodiment of the present disclosure.

FIG. 5 is a diagram showing another embodiment of a probe according to an embodiment of the present disclosure.

FIG. 6 is a diagram showing another embodiment of a probe according to an embodiment of the present disclosure.

FIG. 7 is a diagram depicting a probe and processor according to another embodiment of the present disclosure.

FIG. 8 is a diagram showing a probe according to another embodiment of the present disclosure.

FIG. 9A is an illustration showing a user interface of a localization system according to another embodiment of the present disclosure.

FIG. 9B is an illustration showing a user interface of a localization system according to another embodiment of the present disclosure.

### DETAILED DESCRIPTION OF THE DISCLOSURE

Embodiments of the present disclosure may provide a real-time 3D magnet positional information system that accounts for magnet anisotropy of the magnet. A magnet's field strength may be modeled at any position if the location, orientation, size, shape, and magnetization of the magnet is known. However, the reverse does not apply. In other words, given a magnetic field sample, there is no direct equation for calculating the location of the source magnet, even if its size, shape, and magnetization are known. The present disclosure provides embodiments of a probe designed to locate a hidden magnet source (magnetic marker) using measurements from a discrete magnetic sensor array. By design, the target magnet's size, shape, and magnetization are known, and the remaining parameters (location and orientation/anisotropy) can be estimated using a variety of numerical methods. In some embodiments, this disclosure provides a probe with sensor arrays designed such that:
1. They are sufficiently spaced to provide enough information to resolve the 5D coordinates of the marker.
2. The sensor arrangement is constrained to a 1 cm outer diameter, this is driven by most surgical applications requiring smaller incisions, which necessitates probes to be able to fit in tiny cavities. Additionally, it will also help with laparoscopic procedures, where the trocars used to introduce instruments are on the order of 1 cm in diameter.
3. Provide sufficient information in order to be able to handle two or more markers in the space.

A typical method to determine the solution for such a linear systems of equations is using a gradient descent algorithm such as the one described below. Here r_{i,j} represents the position in cartesian coordinates as well as the angular pose of the marker i in terms of pitch and yaw, with respect to the magnetometer j. M refers to the set of magnetometer measurements, where M(x, y, z) represents a collection of magnetic field measurements at a particular point. B_{i,j}(r) is the calculated magnetic field based on the magnetic dipole moment (m) of marker i with respect to the position of magnetometer j. μ₀ is the magnetic permeability of free space (a constant).

$${r}_{i , j} = [ x , y , x , θ , ϕ ] \qquad ( 1 )$$

$${M}_{j} ( x , y , z ) = {\sum }_{i = 1}^{\# of markers} {B}_{i , j} ( {r}_{i , j} ) \qquad ( 2 )$$

$$[ \begin{matrix}{M}_{1} \\ ⋮ \\ {M}_{n}\end{matrix} ] = [ \begin{matrix}\frac{{μ}_{0}}{4 π} ( \frac{3 {\hat{r}}_{1 , 1} ( {\hat{r}}_{1 , 1} \cdot m ) - m}{{\vert {r}_{1 , 1} \vert}^{3}} ) \\ ⋮ \\ \frac{{μ}_{0}}{4 π} ( \frac{3 {\hat{r}}_{1 , n} ( {\hat{r}}_{1 , n} \cdot m ) - m}{{\vert {r}_{1 , n} \vert}^{3}} )\end{matrix} ] \qquad ( 3 )$$

$$M - B ( r ) = 0 \qquad ( 4 )$$

One example of determining r, would be to minimize the following cost/loss function F(r). Where F(r) will tend towards 0, when the exact position and pose of the marker is determined.

$$F ( r ) = {‖ M - B ( r ) ‖}^{2} \qquad ( 5 )$$

$${min}_{r ∈ ℝ} F ( r ) \to 0 \qquad ( 6 )$$

If the loss/cost function does not converge towards a minima, this is indicative of a noisy environment and can be used as a flag to warn users of potential sources a spurious magnetic signals.

The problem may be set up with more degrees of constraint than degrees of freedom to avoid singularities. Each additional marker pose adds five degrees of freedom to the search problem, and each additional sensor offers three degrees of constraint.

Minimizing the above cost function can be achieved using the gradient descent algorithm. This algorithm iteratively evaluates the cost function and changes the search parameters in the direction of greatest negative gradient. The algorithm stops when the gradient reaches a value close to zero.

The above provides an illustrative technique for determining position and pose of the marker(s). This example is intended be non-limiting, and other techniques may be used and are within the scope of the present disclosure.

### Sensor Layout

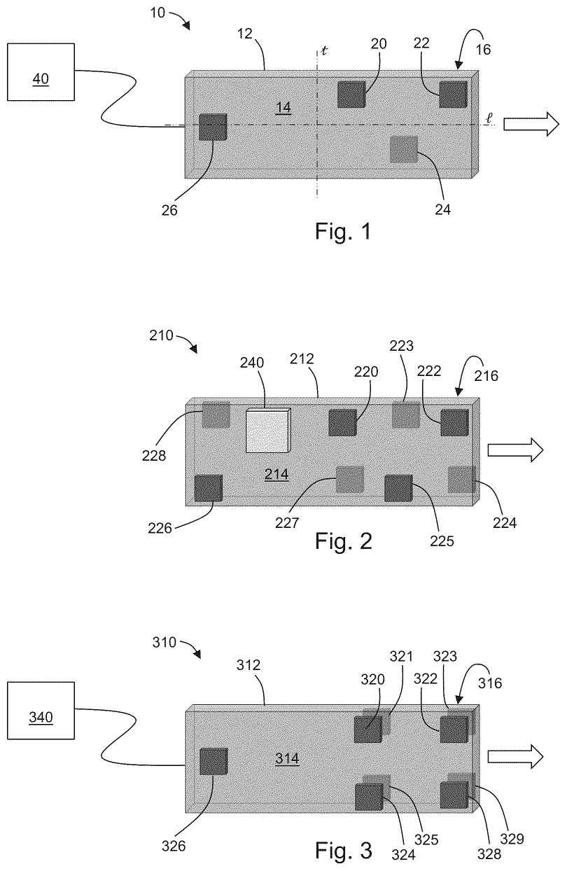

The sensor array geometry (locations and spacings) feeds into the system's localization accuracy. Array configurations with 3-D sensor distributions (i.e., non-coplanar arrangements) are provided to reduce ambiguities and singularities around the detector probe. A minimum number of sensors is required to determine/track the magnet's 3D position and pose. In general, more sensors are better than fewer sensors, but with diminishing accuracy improvements. The algorithms can all be scaled to the number of sensors, at the cost of the added computational burden. Some configurations that have been explored have between 3 and 16 sensors, in various arrangements defining a 3D array. Exemplary embodiments are illustrated in FIGS. 1-6 and 8 and further described below. The sensor spacing is constrained to maintain a probe diameter of less than 12 mm, and preferably 10 mm in diameter as a maximum outer dimension. Minimizing the dimension enables use for minimally-invasive surgeries require which utilize small incisions. These incisions are on the order of 10 mm. The challenge is creating a probe system that provides sufficient information to accurately localize markers within such a small form factor. Examples of magnetic sensors may include inductive coil sensors, magnetoresistive sensors, Hall-effect sensors, and/or any other magnetic sensor suitable for determining a disposition of the magnetic marker.

With reference to FIG. 1, the present disclosure may be embodied as a probe 10 for determining a position of an anisotropic magnetic marker. The magnetic marker has a size, shape, and magnetization that is known—e.g., pre-determined, measured, otherwise obtained, etc. The probe 10 has a substrate 12 with a first side 14 and a second side 16. The substrate 12 has a longitudinal axis l and a transverse axis t perpendicular to the longitudinal axis (such that the longitudinal and transverse axes are on a plane parallel to the first side 14 and/or the second side 16). In some embodiments, the substrate has a thickness of between 0.5 mm and 10 mm, inclusive. In some embodiments, the substrate has a thickness of between 0.8 mm and 1.2 mm, inclusive, such as, for example, 1.0 mm. The probe includes a first magnetic sensor 26 disposed on the first side 14 of the substrate 12. The first magnetic sensor may be a multidimensional magnetic sensor having measurement axes in more than one dimension, such as, for example, a 3-dimensional (3D) magnetic sensor having three orthogonal measurement axes.

A second magnetic sensor 20 is disposed on the first side 14 of the substrate 12 and spaced apart from the first magnetic sensor 26 along the longitudinal axis and the transverse axis. The second magnetic sensor 20 may be a multidimensional magnetic sensor having measurement axes in more than one dimension, such as, for example, a 3D magnetic sensor. In some embodiments, the spacing between the first magnetic sensor and the second magnetic sensor is greater along the longitudinal axis than the spacing between the first magnetic sensor and the second magnetic sensor along the transverse axis.

A third magnetic sensor 24 is disposed on the second side 16 of the substrate 12. The third magnetic sensor 24 may be spaced apart from the first magnetic sensor 26 along the longitudinal axis and/or the transverse axis. The third magnetic sensor 24 may be spaced apart from the second magnetic sensor 20 along the longitudinal axis and/or the transverse axis. The second magnetic sensor 20 may be a multidimensional magnetic sensor having measurement axes in more than one dimension, such as, for example, a 3D magnetic sensor. The third magnetic sensor may be spaced apart from the first magnetic sensor along the longitudinal axis. The third magnetic sensor may be spaced apart from the first magnetic sensor along the transverse axis. The third magnetic sensor may be spaced apart from the second magnetic sensor along the longitudinal axis. The third magnetic sensor may be spaced apart from the second magnetic sensor along the transverse axis.

In some embodiments, at least one of the first magnetic sensor, the second magnetic sensor, and the third magnetic sensor is oriented such that no measurement axis is parallel with the longitudinal axis of the substrate. For example, the first magnetic sensor may be rotated on the first side of the substrate such that the measurement axes are not parallel to either of the longitudinal axis and the transverse axis. Where more than one of the magnetic sensors are oriented in this manner, they may be similarly oriented (e.g., such that the corresponding measurement axes of such magnetic sensors are parallel to each other) or they may be differently oriented (e.g., such that the corresponding measurement axes of such magnetic sensors are not parallel to each other).

In some embodiments, a maximum total spacing between the first magnetic sensor, the second magnetic sensor, and the third magnetic sensor along the transverse axis is less than or equal to 12 mm. In some embodiments, the spacing between the outermost magnetic sensors (including the first magnetic sensor, the second magnetic sensor, the third magnetic sensor, and any additional magnetic sensors (further described below)) is less than or equal to 12 mm. For example, in some embodiments, the total spacing between the first magnetic sensor, the second magnetic sensor, and the third magnetic sensor along the transverse axis does not exceed 10 mm. In some embodiments, the maximum total spacing between the first magnetic sensor, the second magnetic sensor, and the third magnetic sensor (and additional magnetic sensors, if any) along the longitudinal axis is between 1.25 and 10 times the maximum total spacing along the longitudinal axis.

The probe may have one or more additional magnetic sensors on either of the first side or the second side of the substrate (or both where more than one additional magnetic sensors are presented). For example, probe 10 of FIG. 1 includes a fourth magnetic sensor 22 disposed on the first side 14 of the substrate 12. One or more of the additional magnetic sensors, if any, may be multidimensional magnetic sensors, such as, for example, 3D magnetic sensors. The additional magnetic sensor(s) may be spaced apart from one or more of the first magnetic sensor, the second magnetic sensor, and the third magnetic sensor along the longitudinal axis and/or the transverse axis. For example, fourth magnetic sensor 22 of FIG. 1 is spaced apart from the first magnetic sensor 26 along both of the longitudinal axis and the transverse axis, spaced apart from the second magnetic sensor 20 along the longitudinal axis, and spaced apart from the third magnetic sensor 24 along both of the longitudinal axis and the transverse axis.

The probe may have a background magnetic sensor spaced apart from the first magnetic sensor, the second magnetic sensor, the third magnetic sensor, and any additional magnetic sensors. The background magnetic sensor is spaced apart from the other magnetic sensors along (at least) the longitudinal axis. The background magnetic sensor is configured to measure (i.e., detect) a background magnetic field—for example, measure only the background (ambient) magnetic field. For example, the background magnetic sensor may be located such that when a field of a magnetic marker is detectable by the first, second, and/or third magnetic sensors, such marker field is not detectable by the background magnetic sensor (i.e., the field detected by the background magnetic sensor is de minimis). In this way, the background magnetic field may be removed from the field detected by the first, second, and third magnetic sensors (and additional magnetic sensors, if any)—i.e., background rejection. Such background fields may result from the earth's magnetic field, stray magnetic fields from nearby materials and devices, etc. The background magnetic sensor may be at a location more distal from the tip of the probe than the other sensors. In this way, the background magnetic sensor is at a location far enough away from the probe tip so as to not detect a marker's field when the tip of the probe is near the marker. The processor may be configured to remove the field detected by the background magnetic sensor. For example, the processor may subtract the field detected by the background magnetic sensor from the field(s) detect by the other magnetic sensors. In some embodiments, one of the first magnetic sensor, the second magnetic sensor, or the third magnetic sensor is spaced apart from the other magnetic sensors and configured as a background magnetic sensor (i.e., configured to measure a background magnetic field).

Any magnetic sensor on the probe may detect both the background (ambient) magnetic field and the magnetic field of the magnetic marker. Thus, subtracting the field detected by one magnetic sensor from the field detected by any other magnetic sensor on the probe can result in a differential field in which the background magnetic field is removed. For example, with reference to FIG. 1, each of the second sensor 20 and the third sensor 24 can measure both the background magnetic field and the magnetic field of the magnetic marker. Further, when the distal end of the probe 10 is close to the magnetic marker, the third magnetic sensor 24 (which is located closer to the distal end of the probe 10) may measure more of the magnetic field generated by the magnetic marker than the second magnetic sensor 20 measures. Thus, subtracting the field detected by the second magnetic sensor 20 from the field detected by the third magnetic sensor 24 results in a differential field that can be attributed solely to the magnetic marker. In some examples, a plurality of differential fields can be obtained by subtracting the fields detected by neighboring magnetic sensors on the probe (e.g., subtracting the field detected by the third magnetic sensor 24 from the field detected by the fourth magnetic sensor 16, subtracting the field detected by the second magnetic sensor 20 from the field detected by the third magnetic sensor 24, subtracting the field detected by the first magnetic sensor 26 from the field detected by the second magnetic sensor 20). In each resulting differential field, the background magnetic field has been subtracted out. The plurality of differential fields can be then used for downstream processing to determine the disposition of the magnetic marker (e.g., using a machine learning model as described herein). In the example above, each sensor on the probe operates as both a background sensor and an active sensor, as the measurements from each sensor are used both to remove the background magnetic field and to calculate the disposition of the magnetic marker.

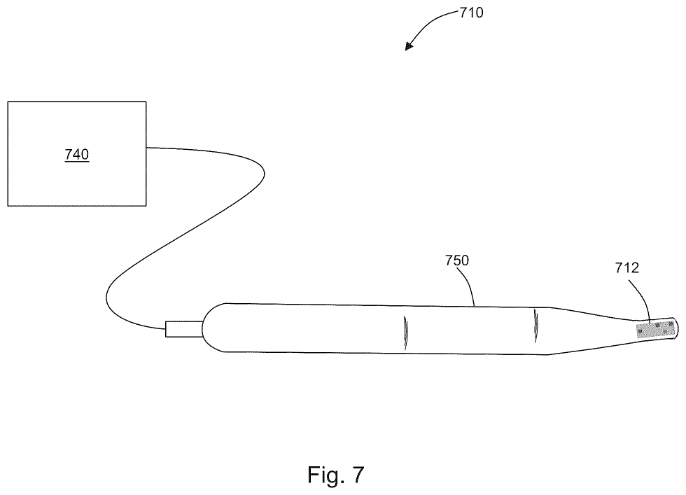

The probe 10 includes a processor 40 in electronic communication with each magnetic sensor (e.g., first magnetic sensor, second magnetic sensor, and third magnetic sensor). The processor may be contained within a housing of a probe. For example, the processor may be disposed on the substrate which is contained within a housing of the probe. In other embodiments, the processor is located outside of the housing of the probe. For example, the processor may be remotely located and in communication with the sensors via wired or wireless connection. FIG. 1 depicts a processor 40 which is located outside of a housing (not shown) of the probe. FIG. 7 shows an example of a probe 710 having a processor 740 located outside of a housing 750. Substrate 712 is shown contained within the housing 750.

The processor is configured to determine a disposition of a magnetic marker in at least five degrees of freedom (5 DoF). The disposition of the magnetic marker is determined based on its known size, shape, and magnetization, as well as signals received by the processor from each of the first magnetic sensor, the second magnetic sensor, and the third magnetic sensor (and additional magnetic sensors of the probe, if any). The processor may be further configured to disregard magnetic field gradients which are not consistent with the magnetic field gradients of the magnetic marker (i.e., the a priori known magnetic field of the magnetic marker). The processor may be configured to determine more than one disposition of the magnetic marker over time. For example, the processor may be configured to periodically determine the disposition of the magnetic marker at a sampling frequency.

In some embodiments, the processor may be configured to determine the disposition of the magnetic marker using machine learning (e.g., a machine-learning classifier). For example, the processor may be configured to determine the disposition using a neural network, such as, for example, a convolution neural network (although other classifiers may be used in addition to or instead of these non-limiting examples). The machine learning classifier (e.g., neural network, etc.) may be trained to determine a disposition of a magnetic marker (for example, a 5DoF disposition of a magnetic marker having known parameters (e.g., magnetic field strength, magnetic field shape, etc.)) using a training set of measurements obtained using a training probe having sensors in a known configuration (e.g., a same configuration as the probe used in clinic). The classifier may be trained using actual measurements, in silico using a derived training set, or a combination of these or other training techniques.

In some embodiments, the processor may be configured to have multiple operating modes. Magnetic field strength falls off dramatically with distance. Farther away from a magnet, the signals are much weaker, and the system's estimations will be littered with noise. This is a result of a low signal-to-noise ratio at the farther-away sensors, which could result in marker positions that cannot be resolved accurately and unambiguously. In these cases, it may be preferred to change modes to a simpler 1D gradient-localization mode, which can be achieved with just one magnetic sensor. The localization mode could be set up to be changed via user input, or it could be done automatically. The processor may be configured with a first mode in which the disposition of the magnetic marker is determined in five degrees of freedom and a second mode wherein the disposition of the magnetic marker is determined using a subset of the available magnetic sensors—for example, a single magnetic sensor.

FIG. 2 depicts another embodiment of a probe 210 having a substrate 212 with a first side 214 and a second side 216. The probe 210 has a first magnetic sensor 226 disposed on the first side of the substrate. The probe 210 has a second magnetic sensor 220 disposed on the first side of the substrate. The probe 210 has a third magnetic sensor 224 disposed on the second side of the substrate. The probe 210 has a fourth magnetic sensor 222 and a fifth magnetic sensor 225, each disposed on the first side of the substrate. The probe 210 has a sixth magnetic sensor 223, a seventh magnetic sensor 227, and an eighth magnetic sensor 228, each disposed on the second side of the substrate. A processor 240 is in electronic communication with each of the first, second, third, fourth, fifth, sixth, seventh, and eighth magnetic sensors.

FIG. 3 depicts another embodiment of a probe 310 having a substrate 312 with a first side 314 and a second side 316. The probe 310 has a first magnetic sensor 326 disposed on the first side of the substrate. The probe 310 has a second magnetic sensor 320 disposed on the first side of the substrate. The probe 310 has a third magnetic sensor 325 disposed on the second side of the substrate. The probe 310 has a fourth magnetic sensor 322, a fifth magnetic sensor 324, and a sixth magnetic sensor 328, each disposed on the first side of the substrate. The probe 310 has a seventh magnetic sensor 321, an eighth magnetic sensor 323, and a ninth magnetic sensor 329, each disposed on the second side of the substrate. A processor 340 is in electronic communication with each of the first, second, third, fourth, fifth, sixth, seventh, eighth, and ninth magnetic sensors.

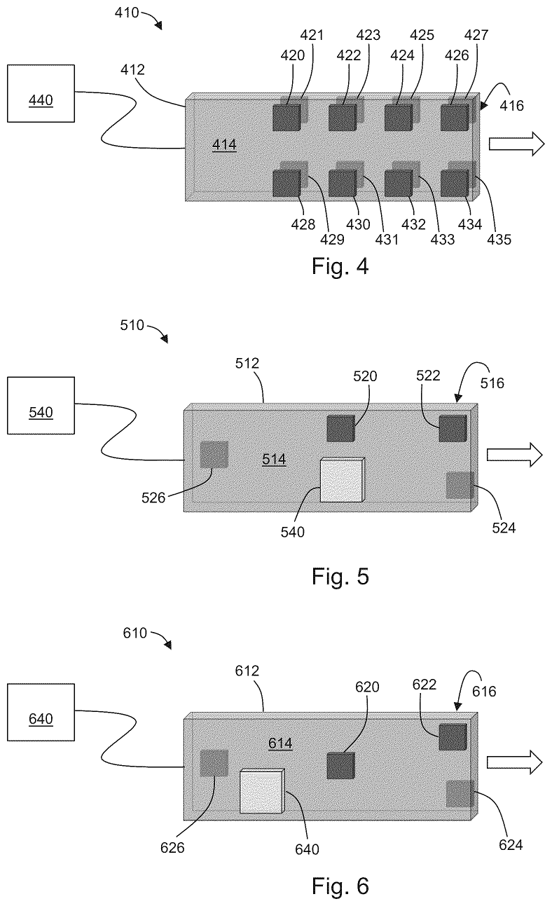

FIG. 4 depicts another embodiment of a probe 410 having a substrate 412 with a first side 414 and a second side 416. The probe 410 has a first magnetic sensor 420 disposed on the first side of the substrate. The probe 410 has a second magnetic sensor 422 disposed on the first side of the substrate. The probe 410 has a third magnetic sensor 421 disposed on the second side of the substrate. The probe 410 has a fourth magnetic sensor 424, a fifth magnetic sensor 426, a sixth magnetic sensor 428, a seventh magnetic sensor 430, an eighth magnetic sensor 432, and a ninth magnetic sensor 434, each disposed on the first side of the substrate. The probe 410 has a tenth magnetic sensor 423, an eleventh magnetic sensor 425, a twelfth magnetic sensor 427, a thirteenth magnetic sensor 429, a fourteenth magnetic sensor 431, a fifteenth magnetic sensor 433, and a sixteenth magnetic sensor 435, each disposed on the second side of the substrate. A processor 440 is in electronic communication with each of the first, second, third, fourth, fifth, sixth, seventh, eighth, ninth, tenth, eleventh, twelfth, thirteenth, fourteenth, fifteenth, and sixteenth magnetic sensors.

FIG. 5 depicts another embodiment of a probe 510 having a substrate 512 with a first side 514 and a second side 516. The probe 510 has a first magnetic sensor 520 disposed on the first side of the substrate. The probe 510 has a second magnetic sensor 522 disposed on the first side of the substrate. The probe 510 has a third magnetic sensor 524 disposed on the second side of the substrate. The probe 510 has a fourth magnetic sensor 526 disposed on the second side of the substrate. A processor 540 is in electronic communication with each of the first, second, third, and fourth magnetic sensors.

FIG. 6 depicts another embodiment of a probe 610 having a substrate 612 with a first side 614 and a second side 616. The probe 610 has a first magnetic sensor 620 disposed on the first side of the substrate. The probe 610 has a second magnetic sensor 622 disposed on the first side of the substrate. The probe 610 has a third magnetic sensor 624 disposed on the second side of the substrate. The probe 610 has a fourth magnetic sensor 626 disposed on the first side of the substrate. In the probe 610 embodiment depicted in FIG. 6, the first magnetic sensor 620 is aligned with the fourth magnetic sensor 626 along the longitudinal axis and spaced apart from the second magnetic sensor 622 along the transverse axis (compare with probe 510 of FIG. 5 where the first magnetic sensor 520 is aligned with the second magnetic sensor 522 along the longitudinal axis and spaced apart from the fourth magnetic sensor 526 along the transverse axis). A processor 640 is in electronic communication with each of the first, second, third, and fourth magnetic sensors.

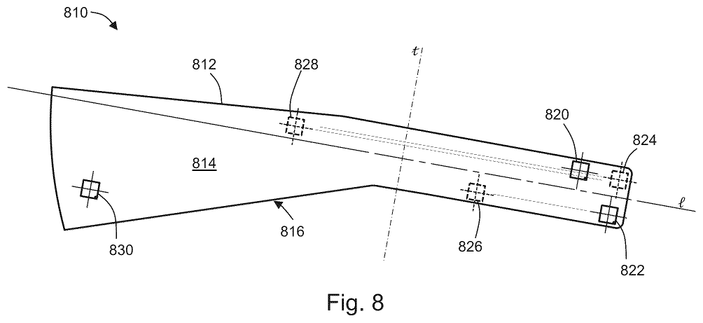

FIG. 8 depicts another embodiment of a probe 810 having a substrate 812 with a first side 814 and a second side 816. The probe 810 has a first magnetic sensor 820 disposed on the first side of the substrate. The probe 810 has a second magnetic sensor 822 disposed on the first side of the substrate. The probe 810 has a third magnetic sensor 824 disposed on the second side of the substrate. The probe 810 has a fourth magnetic sensor 826 disposed on the second side of the substrate. The probe 810 has a fifth magnetic sensor 828 disposed on the second side of the substrate. The probe 810 has a sixth magnetic sensor 830 disposed on the first side of the substrate.

The magnetic sensors 820-830 are spaced apart along the longitudinal axis l of the probe 810. With reference to FIG. 8, the magnetic sensor 824 is the closest to the distal end of the probe 810. The magnetic sensor 822 is the second closest to the distal end of the probe 810 and is spaced apart from the neighboring magnetic sensor 824 along the longitudinal axis l of the probe 810. The magnetic sensor 820 is the third closest to the distal end of the probe 810 and is spaced apart from the neighboring magnetic sensor 822 along the longitudinal axis l of the probe 810. The magnetic sensor 826 is the fourth closest to the distal end of the probe 810 and is spaced apart from the neighboring magnetic sensor 820 along the longitudinal axis l of the probe 810. The magnetic sensor 828 is the fifth closest to the distal end of the probe 810 and is spaced apart from the neighboring magnetic sensor 826 along the longitudinal axis l of the probe 810. The magnetic sensor 830 is the farthest to the distal end of the probe 810 and is spaced apart from the neighboring magnetic sensor 828 along the longitudinal axis l of the probe 810.

In the depicted example, the longitudinal distance between neighboring magnetic sensors increases from the distal end of the probe 810 toward the proximal end of the probe 810. In other words, the magnetic sensors are clustered toward the distal end of the probe 810. For example, the longitudinal distance between the neighboring magnetic sensors 824 and 822 is smaller than the longitudinal distance between the neighboring magnetic sensors 822 and 820, which in turn is smaller than the longitudinal distance between the neighboring magnetic sensors 820 and 826, etc. In some examples, the longitudinal distance between neighboring magnetic sensors from the distal end toward the proximal end of the probe 810 can follow a cubic inverse function, as the magnetic field generated by the magnet marker decreases in strength with respect to the cube of the distance from the magnet marker. The longitudinal spacing can maximize the signal-to-noise ratio and result in more accurate localization of the target magnet.

The magnetic sensors 820-830 are disposed on alternating sides of the probe 810 along the longitudinal axis l of the probe 810. As shown in FIG. 8, the magnetic sensor 824 (i.e., the magnetic sensor closest to the distal end of the probe 810) is disposed on the side 816 of the probe 810. The next two magnetic sensors from the distal end of the probe 810, which are magnetic sensors 822 and 820, are disposed on the opposite side (i.e., side 814) of the probe 810. The next two magnetic sensors from the distal end of the probe 810, which are magnetic sensors 826 and 828, are disposed on the side 816 of the probe 810. In some examples, every two neighboring magnetic sensors may be disposed on opposite sides of the probe 810. For example, neighboring magnetic sensors 822 and 820 may be disposed on opposite sides 814 and 816 of the probe 810 (rather than on the same side as shown in FIG. 8), and neighboring magnetic sensors 826 and 828 may be disposed on opposite sides 814 and 816 of the probe 810 (rather than on the same side as shown in FIG. 8). As another example, in FIG. 1, the magnetic sensor 22 (i.e., the magnetic sensor closest to the distal end of the probe 10) is disposed on the side 14 of the probe 10, the next magnetic sensor 24 is disposed on the side 14 of the probe 10, and the next magnetic sensor 20 is disposed on the side 18 of the probe 10.

Furthermore, magnetic sensors 820-830 are additionally disposed in different positions along the transverse axis t of the probe 810. As shown in FIG. 8, the magnetic sensor 824 (i.e., the magnetic sensor closest to the distal end of the probe 810) is disposed on a first side of the transverse axis t of the probe 810 (i.e., above the longitudinal axis l of the probe 810 in the depicted orientation), the magnetic sensor 822 is disposed on a second side of the transverse axis t of the probe 810 (i.e., below the longitudinal axis l of the probe 810 in the depicted orientation), the magnetic sensor 820 is disposed on the first side of the transverse axis t of the probe 810 (i.e., above the longitudinal axis l of the probe 810 in the depicted orientation), etc.

In the depicted example, the probe 810 comprises a straight section and an angled section. The magnetic sensors 820-826 are located on the straight section. The magnetic sensors 828 and 830 are located on the angled section. As depicted in FIG. 8, in contrast with the straight section, the angled section allows the magnetic sensor 830 to be positioned further way from the longitudinal axis l than the other magnetic sensors, thus providing a larger spatial distribution of the magnetic sensors on the probe 810. The larger spatial distribution of magnetic sensors can result in increased geometric diversity (and thus more precise triangulation), reduced sensitivity to noises (e.g., sensor noise and errors), and higher tolerance for modeling errors, thus allowing more accurate and robust localization of the magnetic marker.

In the probe 810 embodiment depicted in FIG. 8, no magnetic sensor is aligned with another of the magnetic sensors of the probe—i.e., no magnetic sensor has a measurement axis which is co-linear with a measurement axis of another of the magnetic sensors. In other words, the measurement axes of the magnetic sensors are offset from each other (i.e., for 3D magnetic sensors—with respect to the longitudinal axis [symbol] , the transverse axis t, and the z axis (not shown, orthogonal to the view of the figure)). In an exemplary embodiment, a probe having two or more 3D magnetic sensors is configured such that each measurement axis (x, y, z) of the magnetic sensors is parallel to, but not co-linear with, the corresponding measurement axes (x, y, z) of the other magnetic sensors.

The arrangement of the magnetic sensors along the longitudinal and transverse axes in the embodiments disclosed herein can increase or maximize the three-dimensional distribution of the magnetic sensors. The three-dimensional distribution can in turn reduce redundancy in the measurements obtained by the magnetic sensors and thus reduce ambiguity in the processing of the measurements to determine the disposition of the magnetic marker. The disclosed arrangements of the magnetic sensors are particularly advantageous when a limited number of magnetic sensors are included in the system, because it increases or maximizes the information captured by each sensor. In some examples, the probe 810 may include a minimum of four magnetic sensors.

The probe may include a user interface in electronic communication with the processor (e.g., the probe, the processor, and the user interface may make up a localization system or a portion of a localization system). The processor is further configured to provide a signal of the determined disposition of the magnetic marker to the user interface. The user interface may be, for example, a display, such as a computer monitor, or smartphone screen, a tablet screen, etc. In such embodiments, the user interface may display a graphical representation of the marker location according to the signal received form the processor. In some embodiments, the user interface is an audio source. The audio source may be configured to audibly represent the determined disposition of the magnetic marker according to the signal provided from the processor. The user interface may have more than one modality (e.g., both a display and an audio source). In some embodiments, the processor is further configured to provide an indicator signal when magnetic field gradients which are not consistent with the magnetic marker are detected. For example, the processor may signal a display to display a message symbol, or any other indicator or indicia (e.g., color-coding, etc.) to inform a user that a detected signal may not be a magnetic marker.

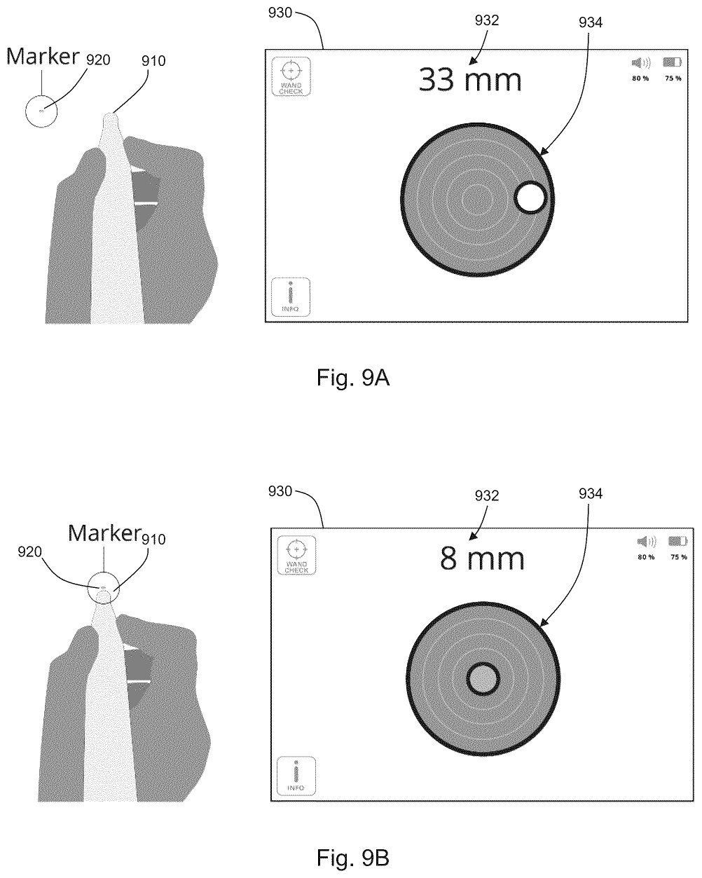

In embodiments wherein the processor determines a disposition of the magnetic marker in sufficient degrees of freedom (e.g., five degrees of freedom), the user interface may provide both distance and position information. For example, the user interface may have a first indicator configured to show a distance to the magnetic marker (e.g., a distance measurement from a tip of the probe to the magnetic marker in mm) and a second indicator configured to show a position of the magnetic marker relative to the probe (left, right, directly in front of, etc.) FIGS. 9A and 9B depict a probe tip 910 position (left) relative to a marker 920, and a corresponding result in a user interface 930 (right). In FIG. 9A, the probe tip 910 is to the right of the marker. The corresponding screen of the user interface 930 has a first indicator 932 showing a distance from the probe tip to the marker (33 mm) and a second indicator 934 showing the probe tip as a white circle indicator to the right of a target center (“bullseye” target) thereby indicating the relative position. In FIG. 9B, the probe tip 910 is oriented directly above the marker 920 (i.e., the marker is in front of the probe tip). In the corresponding screen of the user interface 930, the first indicator 932 shows the distance from the probe tip to the marker (8 mm) and the second indicator 934 shows the probe tip circle at the center of the target. In the embodiment depicted in FIGS. 9A and 9B, the indicator circle showing the probe tip is configured to change color when approaching the center of the target circle (i.e., within a pre-determined distance from the center of the target). Other indicators of distance, position, or both may be used instead of or in addition to these example indicators.

In another aspect, the present disclosure may be embodied as a method of determining the disposition of a magnetic marker. The method includes obtaining a magnetic field strength value of the magnetic marker from each of a plurality of magnetic sensors, where the magnetic sensors have a known configuration relative to one another (e.g., distance and orientation between each of the magnetic sensors). Using a machine-learning classifier of a processor, the disposition of the magnetic marker is determined. In some embodiments, the disposition of the magnetic marker is determined in more than three degrees of freedom (e.g., five degrees of freedom). In some embodiments, one or more of the plurality of magnetic sensors is a multidimensional magnetic sensor. For example, each sensor may be a 3D magnetic sensor (e.g., made up of three magnetometers each oriented along a primary axis—the measurement axes). The machine learning classifier may be a neural network, such as, for example, a convolutional neural network (other classifiers may be used instead of or in addition to these non-limiting examples). The machine learning classifier may be trained to determine a disposition of a magnetic marker (for example, a 5DoF disposition of a magnetic marker having known parameters (e.g., magnetic field strength, magnetic field shape, etc.)) using a training set of measurements using a training probe having sensors in a known configuration (e.g., a same configuration as the probe used in clinic). The classifier may be trained using actual measurements, in silico using a derived training set, or a combination of these or other training techniques.

In some examples, an exemplary system (e.g., one or more electronic devices) receives a set of measurement values (e.g., magnetic field strength values) obtained from a plurality of magnetic sensors on a probe. The plurality of magnetic sensors are arranged on the probe according to a predetermined configuration such as one disclosed herein. In one exemplary implementation, the probe includes six magnetic sensors and each magnetic sensor generates three measurement values (i.e., one measurement value for each measurement axis), thus resulting in 18 measurement values.

In some examples, the system can generate a plurality of input values based on the set of measurement values. For example, the system can subtract a hard iron offset, which represents the magnetic field generated by the measuring magnetic sensor, from each measurement value. Additionally or alternatively, the system can remove the background magnetic field from the set of measurement values. For example, the system can obtain one or more differential fields from the set of measurement values. Each differential field is obtained by subtracting measurement values of two magnetic sensors (e.g., neighboring magnetic sensors). The resulting input data values are provided to the machine-learning model to obtain the disposition of the magnetic marker.

In some examples, the machine learning model is specific to a predetermined configuration or arrangement of magnetic sensors. For example, the machine learning model is trained using data generated by one or more training probes having the same predetermined configuration of magnetic sensors. The training data can include measurement values collected by a training probe when the training probe is in different positions and/or orientations in relation to the magnetic marker, as well as the known dispositions of the magnetic marker. For example, the training data may comprise a plurality of data pairs, each data pair comprising (1) the measurement value(s) collected by a training probe when the training probe is in a particular position or orientation and (2) the associated known disposition of the magnetic marker in relation to the training probe. During training, the machine learning model is configured to receive the measurement value(s) collected by the training probe and predict the disposition of the magnetic marker in relation to the training probe. The predicted disposition is then compared against the ground truth disposition in the training data and the machine learning model can be updated based on the comparison (e.g., using supervised training techniques such as backpropagation). The machine learning model can be iteratively updated based on the plurality of data pairs in the training data to improve the performance of the prediction task. In some examples, a plurality of machine learning models can be trained using training data corresponding to a plurality of predetermined arrangements, respectively. The plurality of machine learning models can be stored on one or more remote devices (e.g., the cloud) accessible over a network. The system can receive, from a probe, measurement values and information identifying the sensor configuration of the probe (e.g., a model number of the probe) and select a machine learning model from the plurality of machine learning models based on the sensor configuration of the probe. The system can then provide measurement values (or input values derived from the measurement values) to the selected machine learning model to obtain the disposition of the marker. The disposition of the magnetic marker can then be transmitted to the probe, a display device, or the like. In some examples, each machine learning model can be trained and retrained iteratively over time using new training data corresponding to the respective predetermined sensor configuration.

In some examples, the machine learning model is specific to an anisotropic geometry. Anisotropic geometry refers to a property of the magnetic field generated by the magnetic marker. For example, the machine learning model is trained using data collected for one or more training magnetic markers that all produce magnetic fields with the same anisotropic geometry. The training data can include a plurality of data pairs, each data pair comprising (1) measurement values collected by a training probe and (2) the associated known disposition of the training magnetic marker. In some examples, a plurality of machine learning models can be trained using training data corresponding to different magnetic fields (e.g., anisotropic geometries), respectively. For example, a first machine learning model can be trained for a first anisotropic geometry of a magnetic field while a second machine learning model can be trained for a second anisotropic geometry of a magnetic field.

In some examples, the machine learning model is specific to an attribute of the magnetic marker (e.g., shapes, sizes, magnetic strengths, models). For example, the machine learning model is trained using data collected for one or more training magnetic markers that all share the same attribute(s). The training data can include a plurality of data pairs, each data pair comprising (1) measurement values collected by a training probe and (2) the associated known disposition of the training magnetic marker. In some examples, a plurality of machine learning models can be trained using training data corresponding to different attributes, respectively. For example, a first machine learning model can be trained for a first magnetic marker model while a second machine learning model can be trained for a second magnetic marker model. As another example, a first machine learning model can be trained for a first magnetic marker shape while a second machine learning model can be trained for a second magnetic marker shape.

The plurality of machine learning models can be stored locally and/or on one or more remote devices (e.g., the cloud) accessible over a network. The system can receive, from a probe, measurement values. The system may further have forehand information identifying the anisotropic geometry of the magnetic field generated by the magnetic marker and select a machine learning model from the plurality of machine learning models based on the anisotropic geometry of the magnetic field. In some examples, the system can infer the anisotropic geometry of the magnetic marker based on the attributes of the magnetic marker. In some examples, the system may have forehand information identifying one or more attributes of the magnetic marker and select a machine learning model from the plurality of machine learning models based on the attribute(s) of the magnetic marker. The system can then provide measurement values (or input values derived from the measurement values) to the selected machine learning model to obtain the location of the magnetic marker. The location of the magnetic marker can then be transmitted to the probe, a display device, or the like. In some examples, each machine learning model can be trained and retrained iteratively over time using new training data corresponding to the respective known anisotropic geometry of a magnetic field or an attribute of the magnetic marker.

In some examples, the machine learning model can be trained to account for magnetic sensor saturation. For example, to account for magnetic sensor saturation, the system can apply a saturation threshold to the simulated training data by capping any measurement over the saturation threshold (e.g., at 50 μT, 500 μT, 2000 μT, 10,000 μT, and the like) to the saturation threshold. Accordingly, the machine learning model can be trained using input measurements that are consistent with real-world magnetic measurements.

The use of machine learning models can provide several technical advantages. At a given time, a probe can generate multiple measurement values from the magnetic sensors. For example, as discussed above, a probe having six magnetic sensors can generate 18 measurement values at any given time. Generating a lookup table for all possible combinations of the 18 measurements would require a large amount of computer storage. Furthermore, using such a lookup table to localize a magnetic marker at real time would be computationally inefficient. In contrast, a machine learning model requires substantially less computer storage to deploy and maintain (either on the probe, on a local device, or on a remote device). Further, the machine learning models allows the system to compress the measurements into a more compact data structure and can generate the estimated location of the magnetic marker more efficiently. Accordingly, the use of machine learning models can improve the functioning of the system by improving its storage, performance, and efficiency.

### Exemplary Embodiments

Among the provided embodiments are:
1. A probe for determining a disposition of a magnetic marker, the probe comprising:
   a substrate having a first side, a second side, a longitudinal axis, and a transverse axis;
   a first magnetic sensor disposed on the substrate;
   a second magnetic sensor neighboring to the first magnetic sensor, the second magnetic sensor disposed on the substrate and spaced further away from a distal end of the substrate than the first magnetic sensor;
   a third magnetic sensor neighboring to the second magnetic sensor, the third magnetic sensor disposed on the substrate and spaced further away from the distal end of the substrate than the second magnetic sensor,
      wherein one of the first magnetic sensor, the second magnetic sensor, and the third magnetic sensor is on the first side of the substrate,
      wherein the other two of the first magnetic sensor, the second magnetic sensor, and the third magnetic sensor are on the second side of the substrate, and
      wherein measurement axes of each of the first magnetic sensor, the second magnetic sensor, and the third magnetic sensor are not co-linear with corresponding measurement axes of the other magnetic sensors; and
   a processor in electronic communication with the first magnetic sensor, the second magnetic sensor, and the third magnetic sensor, wherein the processor is configured to determine the disposition of the magnetic marker based on signals received from each of the first, second, and third magnetic sensors.
2. The probe of embodiment 1, wherein the distance between the first magnetic sensor and the second magnetic sensor along the longitudinal axis of the probe is smaller than the distance between the second magnetic sensor and the third magnetic sensor along the longitudinal axis of the probe.
3. The probe of embodiment 1 or 2, wherein the distance between neighboring magnetic sensors along the longitudinal axis of the probe decreases toward the distal end of the probe.
4. The probe of embodiment 1, 2 or 3, further comprising one or more additional magnetic sensors, wherein every two neighboring magnetic sensors on the probe are located on alternating sides of the substrate.
5. The probe of any of embodiments 1-4, wherein one of the first magnetic sensor, the second magnetic sensor, and the third magnetic sensor is disposed on a first side of the longitudinal axis of the substrate, and wherein the other two of the first magnetic sensor, the second magnetic sensor, and the third magnetic sensor are disposed on a second side of the longitudinal axis of the substrate.
6. The probe of embodiment 5, further comprising one or more additional magnetic sensors, wherein every two neighboring magnetic sensors on the probe are located on different sides of the longitudinal axis of the substrate.
7. The probe of any of embodiments 1-6, wherein the substrate comprises a straight section and an angled section, and wherein at least one of the magnetic sensors on the probe is located on the angled section.
8. The probe of any of embodiments 1-7, wherein the spacing between the first and second magnetic sensors is smaller along the longitudinal axis than the spacing between the first and second magnetic sensors along the transverse axis.
9. The probe of any of embodiments 1-8, wherein a maximum total spacing between the first magnetic sensor, the second magnetic sensor, and the third magnetic sensor along the transverse axis is less than or equal to 12 mm.
10. The probe of any of embodiments 1-9, wherein a maximum total spacing between the first magnetic sensor, the second magnetic sensor, and the third magnetic sensor along the longitudinal axis is between 1.25 and 10 times the maximum total spacing along the longitudinal axis.
11. The probe of any of embodiments 1-10, further comprising a user interface in electronic communication with the processor, and wherein the processor is further configured to provide a signal of the determined disposition of the magnetic marker to the user interface.
12. The probe of any of embodiments 1-11, wherein the processor has a first mode in which the disposition of the magnetic marker is determined in five degrees of freedom and a second mode wherein the disposition of the magnetic marker is determined using one of the first magnetic sensor, the second magnetic sensor, or the third magnetic sensor.
13. The probe of any of embodiments 1-12, wherein the processor is configured to determine more than one disposition of the magnetic marker over time.
14. The probe of any of embodiments 1-13, wherein the processor is configured to periodically determine the disposition of the magnetic marker at a sampling frequency.
15. The probe of any of embodiments 1-14, wherein the substrate is contained within a probe housing.
16. The probe of embodiment 15, wherein the processor is located outside the probe housing.
17. The probe of any of embodiments 1-16, wherein the processor is further configured to provide an indicator signal when magnetic field gradients which are not consistent with the magnetic marker are detected.
18. The probe of any of embodiments 1-17, wherein the processor is further configured to disregard magnetic field gradients which are not consistent with the magnetic marker.
19. The probe of any of embodiments 1-18, wherein at least one of the first magnetic sensor, the second magnetic sensor, and the third magnetic sensor is configured to measure a background magnetic field.
20. The probe of any of embodiments 1-19, further comprising a background magnetic sensor spaced apart from the first magnetic sensor, the second magnetic sensor, and the third magnetic sensor, along the longitudinal axis, and configured to measure a background magnetic field.
21. A method for determining a disposition of a magnetic marker, the method comprising:
   receiving a plurality of magnetic field strength values of the magnetic marker from a plurality of magnetic sensors of a probe, wherein the plurality of magnetic sensors have a pre-determined configuration relative to one another on the probe;
   generating a plurality of input values based on the plurality of magnetic field strength values; and
   providing the plurality of input values to a machine-learning model to obtain the disposition of the marker, wherein the machine-learning model is trained using training data generated by one or more training probes having magnetic sensors in the same pre-determined configuration.
22. The method of embodiment 21, wherein the machine-learning classifier is neural network.
23. The method of embodiment 21 or 22, wherein the machine-learning model is configured to determine the disposition of the magnetic marker in more than three degrees of freedom.
24. The method of embodiment 23, wherein the machine-learning model is configured to determine the disposition of the magnetic marker in five degrees of freedom.
25. The method of any of embodiments 21-24, wherein one or more of the plurality of magnetic sensors is a multidimensional magnetic sensor.
26. The method of embodiment 25, wherein the magnetic field strength values obtained from the one or more multidimensional magnetic sensors are arranged in at least one vector.
27. The method of any of embodiments 21-26, wherein generating the plurality of input values comprises subtracting a hard iron offset from at least one magnetic field strength value of the plurality of magnetic field strength values.
28. The method of any of embodiments 21-27, wherein generating the plurality of input values comprises calculating a differential field by subtracting one or more magnetic field strength values collected by a first magnetic sensor of the plurality of magnetic sensors from one or more magnetic field strength values collected by a second magnetic sensor of the plurality of magnetic sensors.
29. The method of embodiment 28, wherein the first magnetic sensor and the second magnetic sensor are neighboring sensors on the probe.
30. The method of any of embodiments 21-29, wherein the machine-learning model is configured to be retrained iteratively.
31. The method of embodiment 30, wherein the machine-learning model is trained using training data collected for one or more training magnetic markers producing magnetic fields having the same anisotropic geometry as the magnetic marker.
32. The method of any of embodiments 21-31, wherein the probe comprises a substrate having a first side, a second side, a longitudinal axis, and a transverse axis, and wherein the plurality of magnetic sensors comprises a first magnetic sensor disposed on the substrate; a second magnetic sensor neighboring to the first magnetic sensor, the second magnetic sensor disposed on the substrate and spaced further away from a distal end of the substrate than the first magnetic sensor; and a third magnetic sensor neighboring to the second magnetic sensor, the third magnetic sensor disposed on the substrate and spaced further away from the distal end of the substrate than the second magnetic sensor.
33. The method of embodiment 32, wherein the measurement axes of each of the first magnetic sensor, the second magnetic sensor, and the third magnetic sensor are not co-linear with corresponding measurement axes of the other magnetic sensors.
34. The method of embodiment 32 or 33, wherein the spacing between the first and second magnetic sensors is smaller along the longitudinal axis than the spacing between the first and second magnetic sensors along the transverse axis.
35. The method of any of embodiments 21-34, further comprising displaying the determined disposition of the magnetic marker.
36. A non-transitory computer-readable storage medium storing one or more programs, the one or more programs comprising instructions, which when executed by one or more processors of an electronic device having a display, cause the electronic device to:
   receive a plurality of magnetic field strength values of the magnetic marker from a plurality of magnetic sensors of a probe, wherein the plurality of magnetic sensors have a pre-determined configuration relative to one another on the probe;
   generate a plurality of input values based on the plurality of magnetic field strength values; and
   provide the plurality of input values to a machine-learning model to obtain the disposition of the marker, wherein the machine-learning model is trained using training data generated by one or more training probes having magnetic sensors in the same pre-determined configuration.
37. An electronic device, comprising:
   one or more processors,
   a memory, and
   one or more programs, wherein the one or more programs are stored in the memory and configured to be executed by the one or more processors, the one or more programs including instructions for:
      receiving a plurality of magnetic field strength values of the magnetic marker from a plurality of magnetic sensors of a probe, wherein the plurality of magnetic sensors have a pre-determined configuration relative to one another on the probe;
      generating a plurality of input values based on the plurality of magnetic field strength values; and
      providing the plurality of input values to a machine-learning model to obtain the disposition of the marker, wherein the machine-learning model is trained using training data generated by one or more training probes having magnetic sensors in the same pre-determined configuration.
38. A computer program product comprising instructions, which when executed by one or more processors of an electronic device having a display, cause the electronic device to:
   receive a plurality of magnetic field strength values of the magnetic marker from a plurality of magnetic sensors of a probe, wherein the plurality of magnetic sensors have a pre-determined configuration relative to one another on the probe;
   generate a plurality of input values based on the plurality of magnetic field strength values; and
   provide the plurality of input values to a machine-learning model to obtain the disposition of the marker, wherein the machine-learning model is trained using training data generated by one or more training probes having magnetic sensors in the same pre-determined configuration.

Although the present disclosure has been described with respect to one or more particular embodiments, it will be understood that other embodiments of the present disclosure may be made without departing from the spirit and scope of the present disclosure. The following are non-limiting sample claims intended only to illustrate example embodiments.

1. A probe for determining a disposition of a magnetic marker, the probe comprising:
a substrate having a first side, a second side, a longitudinal axis, and a transverse axis;
a first magnetic sensor disposed on the substrate;
a second magnetic sensor neighboring to the first magnetic sensor, the second magnetic sensor disposed on the substrate and spaced further away from a distal end of the substrate than the first magnetic sensor;
a third magnetic sensor neighboring to the second magnetic sensor, the third magnetic sensor disposed on the substrate and spaced further away from the distal end of the substrate than the second magnetic sensor,
wherein one of the first magnetic sensor, the second magnetic sensor, and the third magnetic sensor is on the first side of the substrate,
wherein the other two of the first magnetic sensor, the second magnetic sensor, and the third magnetic sensor are on the second side of the substrate, and
wherein measurement axes of each of the first magnetic sensor, the second magnetic sensor, and the third magnetic sensor are not co-linear with corresponding measurement axes of the other magnetic sensors; and
a processor in electronic communication with the first magnetic sensor, the second magnetic sensor, and the third magnetic sensor, wherein the processor is configured to determine the disposition of the magnetic marker based on signals received from each of the first, second, and third magnetic sensors.

2. The probe of claim 1, wherein the distance between the first magnetic sensor and the second magnetic sensor along the longitudinal axis of the probe is smaller than the distance between the second magnetic sensor and the third magnetic sensor along the longitudinal axis of the probe.

3. The probe of claim 1, wherein the distance between neighboring magnetic sensors along the longitudinal axis of the probe decreases toward the distal end of the probe.

4. The probe of claim 1, further comprising one or more additional magnetic sensors, wherein every two neighboring magnetic sensors on the probe are located on alternating sides of the substrate.

5. The probe of claim 1, wherein one of the first magnetic sensor, the second magnetic sensor, and the third magnetic sensor is disposed on a first side of the longitudinal axis of the substrate, and wherein the other two of the first magnetic sensor, the second magnetic sensor, and the third magnetic sensor are disposed on a second side of the longitudinal axis of the substrate.

6. The probe of claim 5, further comprising one or more additional magnetic sensors, wherein every two neighboring magnetic sensors on the probe are located on different sides of the longitudinal axis of the substrate.

7. The probe of claim 1, wherein the substrate comprises a straight section and an angled section, and wherein at least one of the magnetic sensors on the probe is located on the angled section.

8. The probe of claim 1, wherein the spacing between the first and second magnetic sensors is smaller along the longitudinal axis than the spacing between the first and second magnetic sensors along the transverse axis.

9. The probe of claim 1, wherein a maximum total spacing between the first magnetic sensor, the second magnetic sensor, and the third magnetic sensor along the transverse axis is less than or equal to 12 mm.

10. The probe of claim 1, wherein a maximum total spacing between the first magnetic sensor, the second magnetic sensor, and the third magnetic sensor along the longitudinal axis is between 1.25 and 10 times the maximum total spacing along the longitudinal axis.

11. The probe of claim 1, further comprising a user interface in electronic communication with the processor, and wherein the processor is further configured to provide a signal of the determined disposition of the magnetic marker to the user interface.

12. The probe of claim 1, wherein the processor has a first mode in which the disposition of the magnetic marker is determined in five degrees of freedom and a second mode wherein the disposition of the magnetic marker is determined using one of the first magnetic sensor, the second magnetic sensor, or the third magnetic sensor.

13. The probe of claim 1, wherein the processor is configured to determine more than one disposition of the magnetic marker over time.

14. The probe of claim 1, wherein the processor is configured to periodically determine the disposition of the magnetic marker at a sampling frequency.

15. The probe of claim 1, wherein the substrate is contained within a probe housing.

16. The probe of claim 15, wherein the processor is located outside the probe housing.

17. The probe of claim 1, wherein the processor is further configured to provide an indicator signal when magnetic field gradients which are not consistent with the magnetic marker are detected.

18. The probe of claim 1, wherein the processor is further configured to disregard magnetic field gradients which are not consistent with the magnetic marker.

19. The probe of claim 1, wherein at least one of the first magnetic sensor, the second magnetic sensor, and the third magnetic sensor is configured to measure a background magnetic field.

20. The probe of claim 1, further comprising a background magnetic sensor spaced apart from the first magnetic sensor, the second magnetic sensor, and the third magnetic sensor, along the longitudinal axis, and configured to measure a background magnetic field.
